(19) United States
(12) Patent Application Publication
(10) Pub. No.: US 2005/0154031 A1
(43) Pub. Date: Jul. 14, 2005

(54) 5-Ht2b receptor antagonists

(76) Inventors: Alexander William Oxford, Royston (GB); Richard Anthony Borman, Sawston (GB); Robert Alexander Coleman, Royston (GB); Kenneth Lyle Clark, Linton (GB)

Correspondence Address: NIXON & VANDERHYE, PC, 1100 N GLEBE ROAD, 8TH FLOOR, ARLINGTON, VA, 22201-4714, US

(21) Appl. No.: 10/504,538

(22) PCT Filed: Feb. 11, 2003
(86) PCT No.: PCT/GB03/00567

Related U.S. Application Data
(60) Provisional application No. 60/358,716, filed on Feb. 25, 2002.

(30) Foreign Application Priority Data
Feb. 13, 2002 (JP) 0203413.0

(51) Int. Cl. A61K 31/426

(52) U.S. Cl. 514/370

(57) ABSTRACT

The present invention concerns compounds of formula (I): wherein R¹ is selected from the group consisting of H, and optionally substituted C₁₋₆ alkyl, C₃₋₇ cycloalkyl, C₃₋₇ cycloalkyl-C₁₋₄ alkyl, and phenyl-C₁₋₄ alkyl; R² and R³ are either: (i) independently selected from H, R, R′, SO₂R, C(═O)R, (CH₂)ₙNR⁵R⁶, where n is from 1 to 4 and R⁵ and R⁶ are independently selected from H and R, where R is optionally substituted C₁₋₄ alkyl group, and R′ is an optionally substituted phenyl-C₁₋₄ alkyl group, or (ii) together with the nitrogen atom to which they are attached, form an optionally substituted C₅₋₇ heterocyclic group; R⁴ is an optionally substituted C₉₋₁₄ aryl group; their use as pharmaceuticals, in particular for treating conditions alleviated by antagonism of a 5-HT_{2B} receptor.

This invention relates to 5-HT_{2B} receptor antagonists, pharmaceutical compositions comprising such compounds, and the use of such compounds and compositions to treat various diseases.

### BACKGROUND OF THE INVENTION

Serotonin, also referred to as 5-hydroxytryptamine (5-HT), is a neurotransmitter with mixed and complex pharmacological characteristics. 5-HT acts via a number of discrete 5-HT receptors. Currently, fourteen subtypes of serotonin receptor are recognised and delineated into seven families, 5-HT₁ to 5-HT₇. Within the 5-HT₂ family, 5-HT_{2A}, 5-HT_{2B} and 5-HT_{2C} subtypes are known to exist. The nomenclature and classification of 5-HT receptors has been reviewed by Martin and Humphrey, Neuropharm., 33, 261-273 (1994) and Hoyer, et al., Pharm. Rev., 46, 157-203 (1994).

There is evidence to suggest a role for 5-HT_{2B} receptors in a number of medical disorders, and therefore 5-HT_{2B} receptor antagonists are likely to have a beneficial effect on patients suffering these disorders. They include, but are not limited to: disorders of the GI tract, and especially disorders involving altered motility, and particularly irritable bowel syndrome (WO 01/08668); disorders of gastric motility, dyspepsia, GERD, tachygastria; migraine/neurogenic pain (WO 97/44326); pain (U.S. Pat. No. 5,958,934); anxiety (WO 97/44326); depression (WO 97/44326); benign prostatic hyperplasia (U.S. Pat. No. 5,952,331); sleep disorder (WO 97/44326); panic disorder, obsessive compulsive disorder, alcoholism, hypertension, anorexia nervosa, and priapism (WO 97/44326); asthma and obstructive airway disease (U.S. Pat. No. 5,952,331); incontinence and bladder dysfunction (WO 96/24351); incontinence and bladder dysfunction (WO 96/24351); disorders of the uterus, such as dysmenorrhoea, pre-term labour, post-partum remodelling, endometriosis and fibrosis; pulmonary hypertension (Launay, J. M., et al., Nature Medicine, 8(10), 1129-1135 (2002)).

WO 97/44326 describes aryl pyrimidine derivatives and their use as selective 5-HT_{2B} antagonists. However, although this application discloses a number of compounds, it is desirable to find further classes of compounds to act as 5-HT_{2B} antagonists, which are preferably selective against 5-HT_{2A} and 5-HT_{2C} receptors.

### SUMMARY OF THE INVENTION

A first aspect of the present invention provides the use of a compound of formula I: 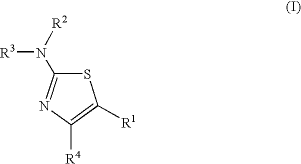
or a pharmaceutically acceptable salt thereof in the preparation of a medicament for the treatment of a condition alleviated by antagonism of a 5-HT_{2B} receptor, wherein R¹ is selected from the group consisting of H, and optionally substituted C₁₋₆ alkyl, C₃₋₇ cycloalkyl, C₃₋₇ cycloalkyl-C₁₋₄ alkyl, and phenyl-C-₁₋₄ alkyl;
R² and R³ are either:
(i) independently selected from H, R, R′, SO₂R, C(═O)R, (CH₂)ₙNR⁵R⁶, where n is from 1 to 4 and R⁵ and R⁶ are independently selected from H and R, where R is optionally substituted C₁₋₄ alkyl group, and R′ is an optionally substituted phenyl-C₁₋₄ alkyl group, or
(ii) together with the nitrogen atom to which they are attached, form an optionally substituted C₅₋₇ heterocyclic group;
R⁴ is an optionally substituted C₉₋₁₄ aryl group;
provided that when R¹ is H, at least two of the fused rings in R⁴ are aromatic or only contain carbon ring atoms.

Conditions which can be alleviated by antagonism of a 5-HT_{2B} receptor are discussed above, and particularly include disorders of the GI tract.

A second aspect of the present invention provides a compound of formula I: 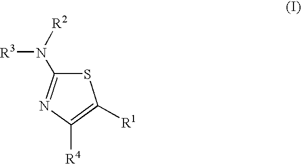
or a pharmaceutically acceptable salt thereof, for use in a method of therapy, wherein
R¹ is selected from the group consisting of H, and optionally substituted C₁₋₆ alkyl, C₃₋₇ cycloalkyl, C₃₋₇ cycloalkyl-C₁₋₄ alkyl, and phenyl-C₁₋₄ alkyl;
R² and R³ are either:
(i) independently selected from H, R, R′, SO₂R, C(═O)R, (CH₂)ₙNR⁵R⁶, where n is from 1 to 4 and R⁵ and R⁶ are independently selected from H and R, where R is a C₁₋₄ alkyl group optionally substituted by hydroxy, alkoxy and amido, and R′ is an optionally substituted phenyl-C₁₋₄ alkyl group, or
(ii) together with the nitrogen atom to which they are attached, form an optionally substituted C₅₋₇ heterocyclic group;
R⁴ is an optionally substituted C₉₋₁₄ aryl group;
provided that when R¹ is H, R² and R³ are independently selected from H and R, and R⁴ is optionally substituted napth-1-yl.

A third aspect of the present invention provides a pharmaceutical composition comprising a compound of formula I as defined in the second aspect or a pharmaceutically acceptable salt thereof together with a pharmaceutically acceptable carrier or diluent.

A fourth aspect of the present invention provides a compound of formula I: 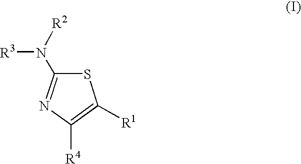
or a salt, solvate or chemically protected form thereof, wherein
R¹ is selected from the group consisting of optionally substituted C₁₋₆ alkyl, C₃₋₇ cycloalkyl, C₃₋₇ cycloalkyl-C₁₋₄ alkyl, and phenyl-C₁₋₄ alkyl;
R² and R³ are either:
(i) independently selected from H, R, R′, SO₂R, C(═O)R, (CH₂)ₙNR⁵R⁶, where n is from 1 to 4 and R⁵ and R⁶ are independently selected from H and R, where R is a C₁₋₄ alkyl group optionally substituted by hydroxy, alkoxy and amido, and R′ is an optionally substituted phenyl-C₁₋₄ alkyl group, or
(ii) together with the nitrogen atom to which they are attached, form an optionally substituted C₅₋₇ heterocyclic group;
R⁴ is an optionally substituted C₉₋₁₄ aryl group

Another aspect of the present invention provides a method of treating a condition which can be alleviated by antagonism of a 5-HT_{2B} receptor, which method comprises administering to a patient in need of treatment an effective amount of a compound of formula I as described in the first aspect of the invention, or a pharmaceutically acceptable salt thereof.

It is preferred that the compounds described above are selective as against 5-HT_{2A} and 5-HT_{2C} receptors.

### DEFINITIONS

C₁₋₆ alkyl group: The term “C₁₋₆ alkyl”, as used herein, pertains to a monovalent moiety obtained by removing a hydrogen atom from a carbon atom of a non-cyclic hydrocarbon compound having from 1 to 6 carbon atoms, and which may be saturated or unsaturated.

Examples of saturated C₁₋₆ alkyl groups include methyl (C₁); ethyl (C₂); propyl (C₃), which may be linear (n-propyl) or branched (iso-propyl); butyl (C₄), which may be linear (n-butyl) or branched (iso-butyl, sec-butyl and tert-butyl); pentyl (C₅), which may be linear (n-pentyl, amyl) or branched (iso-pentyl, neo-pentyl); hexyl (C₆), which may be linear (n-hexyl) or branched.

Examples of unsaturated C₁₋₆ alkyl groups, which may be referred to as C₁₋₆ alkenyl (if they included a double bond) or C₁₋₆ alkynyl (if they include a triple bond) groups, include ethenyl(vinyl, —CH═CH₂), ethynyl(ethinyl, —C≡CH), 1-propenyl(—CH═CH—CH₃), 2-propenyl(allyl, —CH—CH═CH₂), 2-propynyl(propargyl, —CH₂—C≡CH), isopropenyl(—C(CH₃)═CH₂), butenyl (C₄), pentenyl (C₅), and hexenyl (C₆).

C₃₋₇ Cycloalkyl: The term “C₃₋₇ cycloalkyl”, as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 ring atoms

Examples of saturated cycloalkyl groups include, but are not limited to, those derived from: cyclopropane (C₃), cyclobutane (C₄), cyclopentane (C₅), cyclohexane (C₆), and cycloheptane (C₇).

Examples of unsaturated cylcoalkyl groups include, but are not limited to, those derived from: cyclobutene (C₄), cyclopentene (C₅), cyclohexene (C₆), and cycloheptene (C₇).

C₃₋₇ cycloalkyl-C₁₋₄ alkyl: The term “C₃₋₇ cycloalkyl-C₁₋₄ alkyl”, as used herein, pertains to a monovalent moiety obtained by removing a hydrogen atom from a carbon atom of a non-cyclic hydrocarbon compound having from 1 to 4 carbon atoms (C₁₋₄ alkyl), which may be saturated or unsaturated, which itself is substituted by a C₃₋₇ cycloalkyl group.

Examples of C₃₋₇ cycloalkyl-C₁₋₄ alkyl groups include, but are not limited to, those derived from: cyclohexylethane (C₆-C₂) and cyclopentylpropene (C₅-C₃).

Phenyl-C₁₋₄ alkyl: The term “phenyl-C₁₋₄ alkyl”, as used herein, pertains to a monovalent moiety obtained by removing a hydrogen atom from a carbon atom of a non-cyclic hydrocarbon compound having from 1 to 4 carbon atoms (C₁₋₄ alkyl), which may be saturated or unsaturated, which itself is substituted by a phenyl group (C₆H₅—).

Examples of phenyl-C₁₋₄ alkyl groups include, but are not limited to, benzyl(phenyl-CH₂—) and those derived from: phenylethane(phenyl-C₂) and phenylpropene(phenyl-C₃).

C₅₋₇ Heterocyclyl: The term “C₅₋₇ heterocyclyl”, as used herein, pertains to a monovalent moiety obtained by removing a hydrogen atom from a ring atom of a heterocyclic compound, which moiety has from 5 to 7 ring atoms, of which from 1 to 4 are ring heteroatoms. In particular, when R² and R³ together with the nitrogen atom to which they are attached form a C₅₋₇ heterocyclic ring, at least one ring atom will be nitrogen.

Examples of C₅₋₇ heterocyclyl groups having at least one nitrogen atom, include, but are not limited to, those derived from:
N₁: pyrrolidine(tetrahydropyrrole) (C₅), pyrroline (e.g., 3-pyrroline, 2,5-dihydropyrrole) (C₅), 2H-pyrrole or 3H-pyrrole(isopyrrole, isoazole) (C₅), piperidine (C₆), dihydropyridine (C₆), tetrahydropyridine (C₆), azepine (C₇);
N₂: imidazolidine (C₅), pyrazolidine(diazolidine) (C₅), imidazoline (C₅), pyrazoline(dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
N₁O₁S₁: oxathiazine (C₆).

C₉₋₁₄ Aryl: The term “C₉₋₁₄ aryl”, as used herein, pertains to a monovalent moiety obtained by removing a hydrogen atom from an aromatic ring atom of an aromatic compound with at least two fused rings, which moiety has from 9 to 14 ring atoms. Preferably, each ring has from 5 to 7 ring atoms.

The ring atoms may be all carbon atoms, as in “carboaryl groups” (e.g. C₉₋₁₄ carboaryl).

Examples of carboaryl groups include, but are not limited to, those derived from naphthalene (C₁₀), azulene (C₁₀), anthracene (C₁₄) and phenanthrene (C₁₄).

Examples of aryl groups which comprise fused rings, at least one of which is an aromatic ring, include, but are not limited to, groups derived from indene (C₉), isoindene (C₉) tetralin (C₁₀) and fluorene (C₁₃).

Alternatively, the ring atoms may include one or more heteroatoms, as in “heteroaryl groups” (e.g. C₉₋₁₄ heteroaryl).

Examples of heteroaryl groups, include, but are not limited to:
C₉ heteroaryl groups (with 2 fused rings) derived from benzofuran (O₁), isobenzofuran (O₁), indole (N₁), isoindole (N₁), indolizine (N₁), indoline (N₁), isoindoline (N₁), purine (N₄) (e.g. adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiophen (S₁), benzothiazole (N₁S₁), benzothiadiazole (N₂S);
C₁₀ heteroaryl groups (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ heteroaryl groups (with 2 fused rings) derived from benzoazepine (N₁), 5-oxa-9-aza-benzocycloheptene (N₁O₁);
C₁₃ heteroaryl groups (with 3 fused rings) derived from carbazole (N₁), dibenzofuran (O₁), dibenzothiophene (S₁), carboline (N₂), perimidine (N₂), pyridoindole (N₂); and,
C₁₄ heteroaryl groups (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

The above described C₉₋₁₄ aryl group includes the radical formed by removal of a hydrogen atom from any of the possible aromatic ring atoms. The groups formed by this removal can be described by the number of the ring atom from which the hydrogen is removed, if there is more than one possibility. The carboaryl groups derived from, for example, naphthalene (C₁₀) can be either napth-1-yl or nath-2-yl; and from azulene (C₁₀) can be azul-1-yl, azul-2-yl, azul-4-yl, azul-5-yl and azul-6-yl. The heteroaryl groups derived, for example, from isoquinoline can be isoquinol-x-yl(x-isoquinolyl), where x can be 1, 3, 4, 5, 6, 7 or 8.

The phrase “optionally substituted”, as used herein, pertains to a parent group, as above, which may be unsubstituted or which may be substituted by one of the following substituent groups:

C₁₋₂₀ alkyl group: The term “C₁₋₂₀ alkyl”, as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated, partially unsaturated, or fully unsaturated. Thus, the term “alkyl” includes the sub-classes alkenyl, alkynyl and cycloalkyl discussed below.

In this context, the prefixes (e.g. C₁₋₄, C₁₋₇, C₁₋₂₀, C₂₋₇, C₃₋₇, etc.) denote the number of carbon atoms, or range of number of carbon atoms. For example, the term “C₁₋₄ alkyl,” as used herein, pertains to an alkyl group having from 1 to 4 carbon atoms. Examples of groups of alkyl groups include C₁₋₄ alkyl (“lower alkyl”), C₁₋₇ alkyl, and C₁₋₂₀ alkyl.

Examples of saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), n-undecyl (C₁₁), dodecyl (C₁₂), tridecyl (C₁₃), tetradecyl (C₁₄), pentadecyl (C₁₅), and eicodecyl (C₂₀).

Examples of saturated linear alkyl groups include, but are not limited to, methyl (C₁), ethyl (C₂), n-propyl (C₃), n-butyl (C₄), n-pentyl(amyl) (C₅), n-hexyl (C₆), and n-heptyl (C₇) .

Examples of saturated branched alkyl groups include iso-propyl (C₃), iso-butyl (C₄), sec-butyl (C₄), tert-butyl (C₄), iso-pentyl (C₅), and neo-pentyl (C₅) .

Cycloalkyl: The term “cycloalkyl”, as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 20 ring atoms (unless otherwise specified). Preferably, each ring has from 3 to 7 ring atoms.

Examples of saturated cycloalkyl groups include, but are not limited to, those derived from: cyclopropane (C₃), cyclobutane (C₄), cyclopentane (C₅), cyclohexane (C₆), cycloheptane (C₇), norbornane (C₇), norpinane (C₇), norcarane (C₇), adamantane (C₁₀), and decalin(decahydronaphthalene) (C₁₀).

Examples of saturated cycloalkyl groups, which are also referred to herein as “alkyl-cycloalkyl” groups, include, but are not limited to, methylcyclopropyl, dimethylcyclopropyl, methylcyclobutyl, dimethylcyclobutyl, methylcyclopentyl, dimethylcyclopentyl, methylcyclohexyl, and dimethylcyclohexyl, menthane, thujane, carane, pinane, bornane, norcarane, and camphene.

Examples of unsaturated cyclic alkenyl groups, which are also referred to herein as “alkyl-cycloalkenyl” groups, include, but are not limited to, methylcyclopropenyl, dimethylcyclopropenyl, methylcyclobutenyl, dimethylcyclobutenyl, methylcyclopentenyl, dimethylcyclopentenyl, methylcyclohexenyl, and dimethylcyclohexenyl.

Examples of cycloalkyl groups, with one or more other rings fused to the parent cycloalkyl group, include, but are not limited to, those derived from: indene (C₉), indan (e.g., 2,3-dihydro-1H-indene) (C₉), tetraline(1,2,3,4-tetrahydronaphthalene (C₁₀), acenaphthene (C₁₂), fluorene (C₁₃), phenalene (C₁₃), acephenanthrene (C₁₅), aceanthrene (C₁₆). For example, 2H-inden-2-yl is a C₅cycloalkyl group with a substituent (phenyl) fused thereto.

Alkenyl: The term “alkenyl,” as used herein, pertains to an alkyl group having one or more carbon-carbon double bonds. Examples of groups of alkenyl groups include C₂₋₄ alkenyl, C₂₋₇ alkenyl, C₂₋₂₀ alkenyl.

Examples of unsaturated alkenyl groups include, but are not limited to, ethenyl(vinyl, —CH═CH₂), 1-propenyl(—CH═CH—CH₃), 2-propenyl(allyl, —CH—CH═CH₂), isopropenyl(—C(CH₃)═CH₂), butenyl (C₄), pentenyl (C₅), and hexenyl (C₆).

Examples of unsaturated cyclic alkenyl groups, which are also referred to herein as “cycloalkenyl” groups, include, but are not limited to, cyclopropenyl (C₃), cyclobutenyl (C₄), cyclopentenyl (C₅), and cyclohexenyl (C₆).

Alkynyl: The term “alkynyl,” as used herein, pertains to an alkyl group having one or more carbon-carbon triple bonds. Examples of groups of alkynyl groups include C₂₋₄ alkynyl, C₂₋₇ alkynyl, C₂₋₂₀ alkynyl.

Examples of unsaturated alkynyl groups include, but are not limited to, ethynyl(ethinyl, —C≡CH) and 2-propynyl(propargyl, —CH₂—C≡CH).

C₃₋₂₀ heterocyclyl group: The term “C₃₋₂₀ heterocyclyl”, as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆ heterocyclyl,” as used herein, pertains to a heterocyclyl group having 5 or 6 ring atoms. Examples of groups of heterocyclyl groups include C₃₋₂₀ heterocyclyl, C₃₋₇ heterocyclyl, C₅₋₇ heterocyclyl.

Examples of monocyclic heterocyclyl groups include, but are not limited to, those derived from:
N₁: aziridine (C₃), azetidine (C₄), pyrrolidine(tetrahydropyrrole) (C₅), pyrroline (e.g., 3-pyrroline, 2,5-dihydropyrrole) (C₅), 2H-pyrrole or 3H-pyrrole(isopyrrole, isoazole) (C₅), piperidine (C₆), dihydropyridine (C₆), tetrahydropyridine (C₆), azepine (C₇);
O₁: oxirane (C₃), oxetane (C₄), oxolane(tetrahydrofuran) (C₅), oxole(dihydrofuran) (C₅), oxane(tetrahydropyran) (C₆), dihydropyran (C₆), pyran (C₆), oxepin (C₇);
S₁: thiirane (C₃), thietane (C₄), thiolane(tetrahydrothiophene) (C₅), thiane(tetrahydrothiopyran) (C₆), thiepane (C₇);
O₂: dioxolane (C₅), dioxane (C₆), and dioxepane (C₇);
O₃: trioxane (C₆);
N₂: imidazolidine (C₅), pyrazolidine(diazolidine) (C₅), imidazoline (C₅), pyrazoline(dihydropyrazole) (C₅), piperazine (C₆);
N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);
N₁S₁: thiazoline (C₅), thiazolidine (C₅), thiomorpholine (C₆);
N₂O₁: oxadiazine (C₆);
O₁S₁: oxathiole (C₅) and oxathiane(thioxane) (C₆); and,
N₁O₁S₁: oxathiazine (C₆).

Halo: —F, —Cl, —Br, and —I.

Hydroxy: —OH.

Ether: —OR, wherein R is an ether substituent, for example, a C₁₋₇alkyl group (also referred to as a C₁₋₇alkoxy group, discussed below), a C₃₋₂₀heterocyclyl group (also referred to as a C₃₋₂₀heterocyclyloxy group), or a C₅₋₂₀aryl group (also referred to as a C₅₋₂₀aryloxy group), preferably a C₁₋₇alkyl group.

C₁₋₇alkoxy: —OR, wherein R is a C₁₋₇alkyl group. Examples of C₁₋₇alkoxy groups include, but are not limited to, —OMe(methoxy), —OEt(ethoxy), —O(nPr)(n-propoxy), −O(iPr)(isopropoxy), —O(nBu)(n-butoxy), —O(sBu) (sec-butoxy), —O(iBu)(isobutoxy), and —O(tBu) (tert-butoxy).

Oxo(keto, -one): ═O.

Thione(thioketone): ═S.

Imino(imine): ═NR, wherein R is an imino substituent, for example, hydrogen, C₁₋₇alkyl group, a C₃₋₂₀heterocyclyl group, or a C₅₋₂₀aryl group, preferably hydrogen or a C₁₋₇alkyl group. Examples of imino groups include, but are not limited to, ═NH, ═NMe, ═NEt, and ═NPh.

Formyl(carbaldehyde, carboxaldehyde): —C(═O)H.

Acyl(keto): —C(═O)R, wherein R is an acyl substituent, for example, a C₁₋₇alkyl group (also referred to as C₁₋₇alkylacyl or C₁₋₇alkanoyl), a C₃₋₂₀heterocyclyl group (also referred to as C₃₋₂₀heterocyclylacyl), or a C₅₋₂₀aryl group (also referred to as C₅₋₂₀arylacyl), preferably a C₁₋₇alkyl group. Examples of acyl groups include, but are not limited to, —C(═O)CH₃(acetyl), —C(═O)CH₂CH₃(propionyl), —C(═O)C(CH₃)₃(t-butyryl), and —C(═O)Ph(benzoyl, phenone).

Carboxy(carboxylic acid): —C(═O)OH.

Thiocarboxy(thiocarboxylic acid): —C(═S)SH.

Thiolocarboxy(thiolocarboxylic acid): —C(═O)SH.

Thionocarboxy(thionocarboxylic acid): —C(═S)OH.

Imidic acid: —C(═NH)OH.

Hydroxamic acid: —C(═NOH)OH.

Ester(carboxylate, carboxylic acid ester, oxycarbonyl): —C(═O)OR, wherein R is an ester substituent, for example, a C₁₋₇alkyl group, a C₃₋₂₀heterocyclyl group, or a C₅₋₂₀aryl group, preferably a C₁₋₇alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇alkyl group, a C₃₋₂₀heterocyclyl group, or a C₅₋₂₀aryl group, preferably a C₁₋₇alkyl group. Examples of acyloxy groups include, but are not limited to, —OC(═O)CH₃(acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇alkyl group, a C₃₋₂₀heterocyclyl group, or a C₅₋₂₀aryl group, preferably a C₁₋₇alkyl group. Examples of ester groups include, but are not limited to, —OC(═O)OCH₃, —OC(═O)OCH₂CH₃, —OC(═O)OC(CH₃)₃, and —OC(═O)OPh.

Amido(carbamoyl, carbamyl, aminocarbonyl, carboxamide): —C(═O)NR¹R , wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Acylamido(acylamino): —NR¹C(═O)R², wherein R¹ is an amide substituent, for example, hydrogen, a C₁₋₇alkyl group, a C₃₋₂₀heterocyclyl group, or a C₅₋₂₀aryl group, preferably hydrogen or a C₁₋₇alkyl group, and R² is an acyl substituent, for example, a C₁₋₇alkyl group, a C₃₋₂₀heterocyclyl group, or a C₅₋₂₀aryl group, preferably hydrogen or a C₁₋₇alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

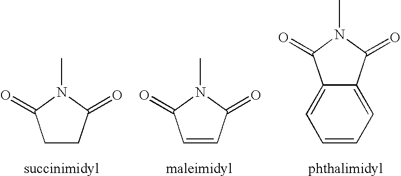

Thioamido(thiocarbamyl): —C(═S)NR¹R², wherein R¹ and R² are independently amino substituents, as defined for amino groups. Examples of thioamido groups include, but are not limited to, —C(═S)NH₂, —C(═S)NHCH₃, —C(═S)N(CH₃)₂, and —C(═S)NHCH₂CH₃.

Ureido: —N(R¹)CONR²R³ wherein R² and R³ are independently amino substituents, as defined for amino groups, and R¹ is a ureido substituent, for example, hydrogen, a C₁₋₇alkyl group, a C₃₋₂₀heterocyclyl group, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

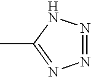

Amino: —NR¹R², wherein R¹ and R² are independently amino substituents, for example, hydrogen, a C₁₋₇alkyl group (also referred to as C₁₋₇alkylamino or di-C₁₋₇alkylamino), a C₃₋₂₀heterocyclyl group, or a C₅₋₂₀aryl group, preferably H or a C₁₋₇alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amidine(amidino): —C(═NR)NR₂, wherein each R is an amidine substituent, for example, hydrogen, a C₁₋₇alkyl group, a C₃₋₂₀heterocyclyl group, or a C₅₋₂₀aryl group, preferably H or a C₁₋₇alkyl group. Examples of amidine groups include, but are not limited to, —C(═NH)NH₂, —C(═NH)NMe₂, and —C(═NMe)NMe₂.

Nitro: —NO₂.

Nitroso: —NO.

Cyano(nitrile, carbonitrile): —CN.

Sulfhydryl(thiol, mercapto): —SH.

Thioether(sulfide): —SR, wherein R is a thioether substituent, for example, a C₁₋₇alkyl group (also referred to as a Cl₇alkylthio group), a C₃-₂₀heterocyclyl group, or a C₅₋₂₀aryl group, preferably a C₁₋₇alkyl group. Examples of C₁₋₇alkylthio groups include, but are not limited to, —SCH₃ and —SCH₂CH₃.

Disulfide: —SS—R, wherein R is a disulfide substituent, for example, a C₁₋₇alkyl group, a C₃₋₂₀heterocyclyl group, or a C₅₋₂₀aryl group, preferably a C₁₋₇alkyl group (also referred to herein as C₁₋₇alkyl disulfide). Examples of C₁₋₇alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇alkyl group, a C₃₋₂₀heterocyclyl group, or a C₅₋₂₀aryl group, preferably a C₁₋₇alkyl group. Examples of sulfine groups include, but are not limited to, —S(═O)CH₃ and —S(═O)CH₂CH₃.

Sulfone(sulfonyl): —S(═O)₂R, wherein R is a sulfone substituent, for example, a C₁₋₇alkyl group, a C₃₋₂₀ heterocyclyl group, or a C₅₋₂₀aryl group, preferably a C₁₋₇alkyl group, including, for example, a fluorinated or perfluorinated C₁₋₇alkyl group. Examples of sulfone groups include, but are not limited to, —S(═O)₂CH₃(methanesulfonyl, mesyl), —S(═O)₂CF₃(triflyl), —S(═O)₂CH₂CH₃(esyl), —S(═O)₂C₄F₉(nonaflyl), —S(═O)₂CH₂CF₃(tresyl), —S(═O)₂CH₂CH₂NH₂(tauryl), —S(═O)₂Ph(phenylsulfonyl, besyl), 4-methylphenylsulfonyl(tosyl), 4-chlorophenylsulfonyl(closyl), 4-bromophenylsulfonyl(brosyl), 4-nitrophenyl(nosyl), 2-naphthalenesulfonate(napsyl), and 5-dimethylamino-naphthalen-1-ylsulfonate(dansyl).

Sulfinic acid(sulfino): —S(═O)OH, —SO₂H.

Sulfonic acid(sulfo): —S(═O)₂OH, —SO₃H.

Sulfinate(sulfinic acid ester): —S(═O)OR; wherein R is a sulfinate substituent, for example, a C₁₋₇alkyl group, a C₃₋₂₀heterocyclyl group, or a C₅₋₂₀aryl group, preferably a C₁₋₇alkyl group. Examples of sulfinate groups include, but are not limited to, —S(═O)OCH₃(methoxysulfinyl; methyl sulfinate) and —S(═O)OCH₂CH₃(ethoxysulfinyl; ethyl sulfinate).

Sulfonate(sulfonic acid ester): —S(═O)₂OR, wherein R is a sulfonate substituent, for example, a C₁₋₇alkyl group, a C₃₋₂₀heterocyclyl group, or a C₅₋₂₀aryl group, preferably a C₁₋₇alkyl group. Examples of sulfonate groups include, but are not limited to, —S(═O)₂OCH₃(methoxysulfonyl; methyl sulfonate) and —S(═O)₂OCH₂CH₃(ethoxysulfonyl; ethyl sulfonate).

Sulfinyloxy: —OS(=O)R, wherein R is a sulfinyloxy substituent, for example, a C₁₋₇alkyl group, a C₃₋₂₀heterocyclyl group, or a C₅₋₂₀aryl group, preferably a C₁₋₇alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇alkyl group, a C₃₋₂₀heterocyclyl group, or a C₅₋₂₀aryl group, preferably a C₁₋₇alkyl group. Examples of sulfonyloxy groups include, but are not limited to, —OS(═O)₂CH₃(mesylate) and —OS(═O)₂CH₂CH₃(esylate).

Sulfate: —OS(═O)₂OR; wherein R is a sulfate substituent, for example, a C₁₋₇alkyl group, a C₃₋₂₀heterocyclyl group, or a C₅₋₂₀aryl group, preferably a C₁₋₇alkyl group. Examples of sulfate groups include, but are not limited to, —OS(═O)₂OCH₃ and —SO(═O)₂OCH₂CH₃.

Sulfamyl(sulfamoyl; sulfinic acid amide; sulfinamide): —S(═O)NR¹R², wherein R¹ and R² are independently amino substituents, as defined for amino groups. Examples of sulfamyl groups include, but are not limited to, −S(═O)NH₂, —S(═O)NH(CH₃), —S(═O)N(CH₃)₂, —S(═O)NH(CH₂CH₃), —S(═O)N(CH₂CH₃)₂, and —S(═O) NHPh.

Sulfonamido(sulfinamoyl; sulfonic acid amide; sulfonamide): —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇alkyl group, a C₃₋₂₀heterocyclyl group, or a C₅₋₂₀aryl group, preferably a C₁₋₇alkyl group. Examples of sulfinamino groups include, but are not limited to, —NHS(═O)CH₃ and —N(CH₃)S(═O)C₆H₅.

The above listed substituent groups may themselves be further substituted, by one or more of themselves.

Includes Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

Isomers, Salts, Solvates and Protected Forms

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r- forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”)

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

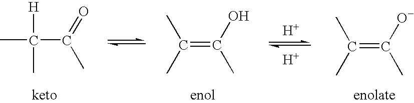

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19, which is incorporated herein by reference.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺and K⁺, alkaline earth cations such as Ca²⁺and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucoheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999), which is incorporated herein by reference.

A wide variety of such “protecting”, “blocking”, or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether(—OR) or an ester(—OC(═O)R), for example, as: a t-butyl ether; a benzyl, benzhydryl(diphenylmethyl), or trityl(triphenylmethyl)ether; a trimethylsilyl or t-butyldimethylsilyl ether; or an acetyl ester(—OC(═O)CH₃, —OAc).

For example, an aldehyde or ketone group may be protected as an acetal(R—CH(OR)₂) or ketal(R₂C(OR)₂), respectively, in which the carbonyl group (>C═O) is converted to a diether(>C(OR)₂), by reaction with, for example, a primary alcohol. The aldehyde or ketone group is readily regenerated by hydrolysis using a large excess of water in the presence of acid.

For example, an amine group may be protected, for example, as an amide(—NRCO—R) or a urethane(—NRCO—OR), for example, as: a methyl amide(—NHCO—CH₃); a benzyloxy amide(—NHCO—OCH₂C₆H₅, —NH-Cbz); as a t-butoxy amide(—NHCO—OC(CH₃)3, —NH-Boc); a 2-biphenyl-2-propoxy amide(—NHCO—OC(CH₃)₂C₆H₄C₆H₅, —NH-Bpoc), as a 9-fluorenylmethoxy amide(−NH-Fmoc), as a 6-nitroveratryloxy amide(—NH-Nvoc), as a 2-trimethylsilylethyloxy amide(—NH-Teoc), as a 2,2,2-trichloroethyloxy amide(-NH-Troc), as an allyloxy amide(—NH-Alloc), as a 2(-phenylsulfonyl)ethyloxy amide(—NH-Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether(—SR), for example, as: a benzyl thioether; an acetamidomethyl ether(—S—CH₂NHC(═O)CH₃).

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e., prophylaxis) is also included.

The term “therapeutically-effective amount,” as used herein, pertains to that amount of an active compound, or a material, composition or dosage from comprising an active compound, which is effective for producing some desired therapeutic effect, commensurate with a reasonable therapeutic effect, commensurate with a reasonable benefit/risk ratio, when administered in accordance with a desired treatment regimen. Suitable dose ranges will typically be in the range of from 0.01 to 20 mg/kg/day, preferably from 0.1 to 10 mg/kg/day.

Compositions and their Administration

Compositions may be formulated for any suitable route and means of administration. Pharmaceutically acceptable carriers or diluents include those used in formulations suitable for oral, rectal, nasal, topical (including buccal and sublingual), vaginal or parenteral (including subcutaneous, intramuscular, intravenous, intradermal, intrathecal and epidural) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound as defined above may be formulated as suppositories using, for example, polyalkylene glycols, acetylated triglycerides and the like, as the carrier. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pennsylvania, 15th Edition, 1975. The composition or formulation to be administered will, in any event, contain a quantity of the active compound(s) in an amount effective to alleviate the symptoms of the subject being treated.

Dosage forms or compositions containing active ingredient in the range of 0.25 to 95% with the balance made up from non-toxic carrier may be prepared.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium carbonate, and the like. Such compositions take the form of solutions, suspensions, tablets, pills, capsules, powders, sustained release formulations and the like. Such compositions may contain 1%-95% active ingredient, more preferably 2-50%, most preferably 5-8%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

Acronyms

For convenience, many chemical moieties are represented using well known abbreviations, including but not limited to, methyl (Me), ethyl (Et), n-propyl (nPr), iso-propyl (iPr), n-butyl (nBu), sec-butyl (sBu), iso-butyl (iBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), acetonitrile (ACN), trifluoroacetic acid (TFA), dimethylformamide (DMF), tetrahydrofuran (THF), and dimethylsulfoxide (DMSO).

General Synthesis Methods

Compounds according to the present invention can be synthesised according to the following route.

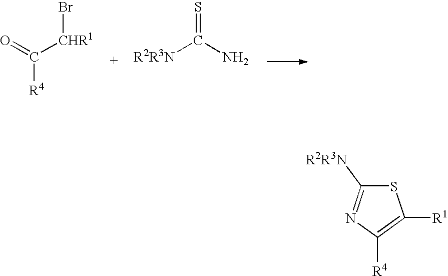

In this method the 2-amino thiazole is produced by the condensation of the appropriate α-bromo ketone with an appropriately substituted thiourea, which reaction is carried out in an organic solvent.

The 5-substituent on the thiazole ring is present in the starting material as the alkyl chain of the α-bromo alkylarylketone, which can be obtained from the parent alkylarylketone if necessary.

The starting ketones for this route are either commercially available or accessible by, for example, Grignard reactions on the corresponding nitriles or Friedal Crafts reaction of substituted aryls.

A further method of preparing compounds of the present invention is by a palladium catalysed coupling reaction of a 2-amino-4-substituted thiazole with an aryl boronic acid, or derivative thereof. The 4-substituent on the thiazole ring may typically be a halogen, such as bromo, iodo or chloro, or a group such as trifluoromethanesulfonate or a phosphate ester. The aryl boronic acid may also be replaced by certain magnesium, tin or zinc containing organometallic reagents. For example, a 2-amino-4-bromo-thiazole may be reacted with an aryl boronic acid derivative in an aqueous solvent, for example a mixture of ethanol, water and dimethoxyethane, containing a palladium catalyst such as tetrakis(triphenylphosphine)palladium(0) and an inorganic base such as sodium carbonate. The reaction is carried out by heating at about 80-90° for several hours.

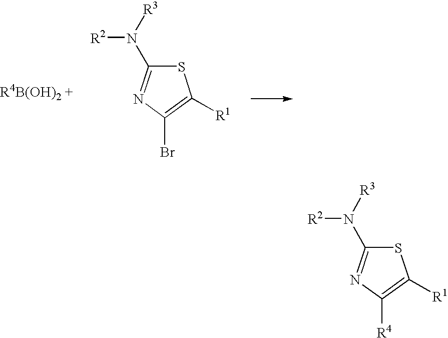

Alternatively, the boronic acid residue, or equivalent, may be on the 4-position of the thiazole ring and the halogen, or equivalent, on the aryl group.

In either of the above routes, any substitution on the aryl group is preferably present in the relevant starting material, but could be introduced later in the reaction scheme, with, if necessary, appropriate protection of other functional groups present in the molecule.

Preferences

The following preferences may be combined with one another, and may be different for each aspect of the present invention.

The optional substituents for R¹, R², R³ and R⁴ are preferably independently selected from halo, hydroxy, alkoxy (more preferably C₁₋₄ alkoxy), amino (more preferably NH₂, C₁₋₄ alkyl amino, C₁₋₄ dialkyl amino), and amido (more preferably CONH₂, C₁₋₄ alkyl amido, C₁₋₄ dialkyl amido)

First Aspect

R¹ is preferably selected from H and optionally substituted C₁₋₆ alkyl and C₃₋₇ cycloalkyl, more preferably H and optionally substituted C₁₋₆ alkyl. Especially preferred are H, and C₁₋₄ alkyl (e.g. methyl, iso-propyl). In some embodiments R¹ may be unsubstituted, but when R¹ is substituted, preferred substituent groups include halo, hydroxy, and amino.

In some embodiments it is preferred that both R² and R³ are substituted, and in other embodiments that only one or neither of R² and R³ are substituted. Each of R² and R³ are preferably independently selected from H, R, R′, where R and R′ are as defined above, and more preferably selected from H and R. R is preferably an optionally substituted C₁₋₄ alkyl group. The preferred substituents for R and R′ include halo, hydroxy, and amino.

It is preferred that all of the fused rings in R⁴ are aromatic or only contain only carbon rings atoms (i.e. a carboaryl group).

R⁴ is preferably an optionally substituted C₉₋₁₄ carboaryl group, for example, naphth-1-yl, naphth-2-yl, anthracen-1-yl, anthracen-2-yl, anthracen-9-yl, phenanthren-1-yl, phenanthren-2-yl, phenanthren-3-yl and phenanthren-4-yl, phenanthren-9-yl. Of these napth-1-yl and napth-2-yl are preferred, with napthy-1-yl being most preferred. Preferred substituent groups for R⁴ include halo, hydroxy, amino, amido and C₁₋₄ alkyl.

Particularly preferred compounds include: 2-amino-5-methyl-4-(naphth-1-yl)thiazole (1), 2-amino-5-isopropyl-4-(naphth-1-yl)thiazole (2); 2-amino-4-(naphth-1-yl)thiazole (3) and 2-amino-4-(naphth-2-yl)thiazole (4).

Second Aspect

R¹ is preferably selected from H and optionally substituted C₁₋₆ alkyl and C₃₋₇ cycloalkyl, more preferably H and optionally substituted C₁₋₆ alkyl. Especailly preferred are H, and C₁₋₄ alkyl (e.g. methyl, iso-propyl). In some embodiments R¹ may be unsubstituted, but when R¹ is substituted, preferred substituent groups include halo, hydroxy, and amino.

In some embodiments it is preferred that both R² and R³ are substituted, and in other embodiments that only one or neither of R² and R³ are substituted.

In R² and R³, R is preferably an optionally substituted C₁₋₄ alkyl group. The preferred substituents for R and R′ include halo, hydroxy, and amino.

Preferred substituent groups for R⁴ include halo, hydroxy, amino, amido and C₁₋₄ alkyl.

When R¹ is not H, each of R² and R³ are preferably independently selected from H, R, R′, where R and R′ are as defined above, and more preferably selected from H and R.

When R¹ is not H, R⁴ is preferably an optionally substituted C₉₋₁₄ carboaryl group, for example, naphth-1-yl, naphth-2-yl, anthracen-1-yl, anthracen-2-yl, anthracen-9-yl, phenanthren-1-yl, phenanthren-2-yl, phenanthren-3-yl and phenanthren-4-yl, phenanthren-9-yl. Of these napth-1-yl and napth-2-yl are preferred, with napthy-1-yl being most preferred.

Particularly preferred compounds include: 2-amino-5-methyl-4-(naphth-1-yl)thiazole (1), 2-amino-5-isopropyl-4-(naphth-1-yl)thiazole (2) and 2-amino-4-(naphth-1-yl)thiazole (3).

Fourth Aspect

R¹ is preferably selected from optionally substituted C₁₋₆ alkyl and C₃₋₇ cycloalkyl, more preferably optionally substituted C₁₋₆ alkyl. Especailly preferred are C₁₋₄ alkyl (e.g. methyl, iso-propyl). In some embodiments R¹ may be unsubstituted, but when R¹ is substituted, preferred substituent groups include halo, hydroxy, and amino.

In some embodiments it is preferred that both R² and R³ are substituted, and in other embodiments that only one or neither of R² and R³ are substituted. Each of R² and R³ are preferably independently selected from H, R, R′, where R and R′ are as defined above, and more preferably selected from H and R. R is preferably an optionally substituted C₁₋₄ alkyl group. The preferred substituents for R and R′ include halo, hydroxy, and amino.

R⁴ is preferably an optionally substituted C₉₋₁₄ carboaryl group, for example, naphth-1-yl, naphth-2-yl, anthracen-1-yl, anthracen-2-yl, anthracen-9-yl, phenanthren-1-yl, phenanthren-2-yl, phenanthren-3-yl and phenanthren-4-yl, phenanthren-9-yl. Of these napth-1-yl and napth-2-yl are preferred, with napthy-1-yl being most preferred. Preferred substituent groups for R⁴ include halo, hydroxy, amino, amido and C₁₋₄ alkyl.

Particularly preferred compounds include 2-amino-5-methyl-4-(naphth-1-yl)thiazole (1) and 2-amino-5-isopropyl-4-(naphth-1-yl)thiazole (2).

Selectivity

The selectivity of the compound for antagonising 5-HT_{2B} receptors over 5-HT_{2A} and/or 5-HT_{2C} receptors can be quantified by dividing the Ki for 5-HT_{2B} (see below) by the Ki for 5-HT_{2A/2C} (see below). The resulting ratio is preferably 10 or more, more preferably 100 or more.

The following examples illustrate the invention.

### EXAMPLE 1

### Synthesis of 2-amino-5-methyl-4-(naphth-1-yl)thiazole (1)

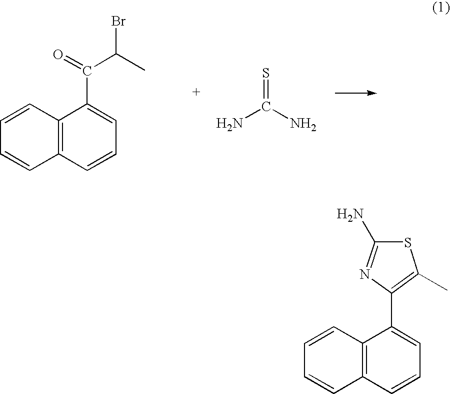

2-Bromo-1-(naphth-1-yl)-propan-1-one (9.5 g) and thiourea (6.2 g) were heated to 100° C. in anhydrous toluene (60 ml) for 2 hours. After cooling, the mixture was evaporated in vacuo and the residue dissolved in methanol (40 ml). Dilute hydrochloric acid (0.5M; 250 ml) was added and the resulting solution was washed twice with ether then basified with sodium hydroxide solution (2M). The mixture was extracted with dichloromethane and chloroform. The combined organic extracts were washed with water, dried with sodium sulphate, filtered and evaporated in vacuo. The title compound (1) (4.45 g, m.p. 194-195° C.) was obtained following re-crystallisation of the residue in ethyl acetate.

¹H NMR (CDCl₃, δ): 2.2 (3H, s); 5.0 (2H, broad s); 7.5 (4H, m); 7.9 (2H, m)

Mass spectrum (m/z): 241 (M+H)⁺

Microanalysis: C, expected 69.97; found 70.86; H, expected 5.03; found 5.03; N, expected 11.66; found 11.17;

### EXAMPLE 2

### Synthesis of 2-amino-5-isopropyl-4-(naphth-1-yl)thiazole (2)

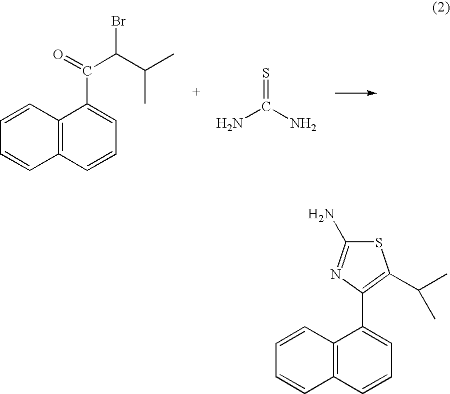

2-Bromo-3-methyl-1-(naphth-1-yl)butan-1-one (4.5g) and thiourea (5.9 g) were heated to 105° C. in anhydrous dimethylformamide (15 ml) for 24 hours. After cooling, the mixture was added to sodium bicarbonate solution and extracted twice with ethyl acetate. The combined organic extracts were washed with water, brine, dried with sodium sulphate, filtered and evaporated in vacuo. The residue was dissolved in ether and extracted twice with hydrochloric acid (2M). The combined aqueous extracts were basified with sodium hydroxide solution (2M) and the resulting mixture was extracted with dichloromethane. The organic extract was dried with sodium sulphate, filtered and evaporated in vacuo. The title compound (2) was obtained as a foam (0.18 g) following silica gel column chromatography of the residue in 0-1.5% methanol in dichloromethane then 33% ethyl acetate in petroleum ether.

¹H NMR (CDCl₃, δ): 1.2 (3H, s); 2.95 (1H, septet); 4.8 (2H, broad s); 7.5 (4H, m); 7.9 (3H, m)

Mass spectrum (m/z): 269 (M+H)⁺

Microanalysis: C, expected 71.61; found 71.45; H, expected 6.01; found 6.11; N, expected 10.44; found 10.02;

### EXAMPLE 3

### 2-amino-4-(naphth-1-yl)thiazole (3) and 2-amino-4-(naphth-2-yl)thiazole (4)

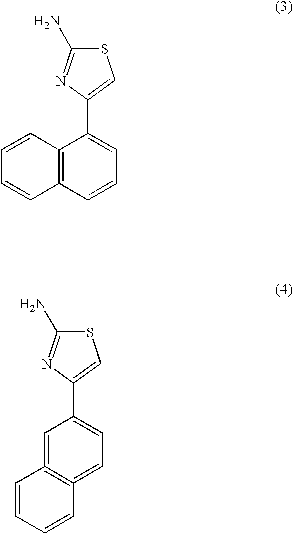

These compounds were obtained from Lancaster Synthesis UK (Morecambe, Lancashire, UK) for testing in the subsequently described assays.

Human Cloned 5-HT_{2B} Receptor Binding Assay

The binding affinity of the compounds for human cloned 5-HT_{2B} receptors was determined using the following assay.

CHO-K1 cells expressing cloned 5-HT_{2B} receptor were maintained in Ultra-CHO medium containing 400 μg/ml of G418, 100 U/ml penicillin, 100 μg/ml streptomycin, 2.5 μg/ml fungizone and 1% foetal bovine serum, in 95/5% O₂/CO₂ at 37° C. The cells were harvested using 0.25% trypsin and were centrifuged at 800 rpm for 8 minutes. The cells were homogenised in 50 mM HEPES buffer containing 1 mM disodium EDTA and 1 mM PMSF at pH 7.4, using a Dounce homogeniser (20 strokes). The homogenate was centrifuged at 2280 rpm (1000 g) and 4° C. for 10 minutes, after which the supernatant was removed by decanting. The pellet was re-homogenised as above, and the resulting supernatant removed and combined with that already obtained. The supernatant solution was then centrifuged at 18300 rpm (40000 g) for 10 minutes at 4° C. using a Sorvall centrifuge. The supernatant was removed, and the pellet was re-suspended in 50 mM buffer at pH 7.4 using a Ultra-turrax T25 Polytron, before centrifugation again at 40000 g as above. This wash procedure was repeated, after which the membrane preparation was stored at a concentration of 1 mg/ml at −80° C. until use.

The membranes were thawed rapidly and diluted in assay buffer containing Tris-HCl (50 mM, pH 7.4), ascorbic acid (0.1%) and calcium chloride (4 mM). The membranes were homogenised to resuspend them, prior to adding 10 or 15 μg of membranes to assay wells containing [³H]LSD (1 nM), assay buffer (50 mM Tris, 4 mM calcium chloride and 0.1% ascorbic acid) containing pargyline (10 μM), and the test compounds (1×10⁻¹⁰ to 1×10⁻⁴ M). Non specific binding was determined in the presence of 100 μM 5-HT. After 30 minutes incubation at 37° C., the assay mixture was filtered through a combination of GF-C and GF-B filters, pre-soaked in 1% polyethyleneimine, using a Brandel cell harvester, and were washed three times using 50 mM Tris-HCl. Radioactivity retained on the filters was determined by liquid scintillation counting. For each test compound, the concentration that inhibited binding of [³H)LSD by 50% was determined using curve fitting software (Prism). Kd values (concentration of LSD required to occupy 50% of the receptor binding sites at equilibrium) determined from saturation binding studies were then used to calculate inhibition dissociation constants (Ki) using the following equation:

$$Ki = \frac{{IC}_{50}}{1 + ( \frac{Radioligand concentration}{Radioligand Kd} )}$$

The results are shown in table 1 below as pKi values. This approach follows that set out in Kenakin, T. P. Pharmacologic analysis of drug-receptor interaction. Raven Press, New York, 2^{nd} Edition, which is incorporated herein by reference.

Human 5-HT_{2A} and 5-HT_{2C} Receptor Binding Assays

The binding affinity of ligands for human 5-HT_{2A} and 5-HT_{2C} receptors was determined using the following assay. These results were then used to determine the selectivity of the test compounds for 5-HT_{2B} receptors, over 5-HT_{2A} and 5-HT_{2C} receptors.

Membrane preparations from CHO-K1 cells expressing the cloned human 5-HT_{2A} receptor were obtained (Euroscreen). The membranes were thawed rapidly and diluted in assay buffer containing Tris-HCl (50 mM, pH 7.7). The membranes were resuspended by homogenisation, prior to adding 15 μg of membranes to assay wells containing [³H] ketanserin (1 nM), assay buffer (50 mM Tris at pH 7.4) containing pargyline (10 μM), and test compounds (1×10⁻¹⁰ to 1×10⁻⁴M). Non specific binding was determined in the presence of 100 μM mianserin. After 15 minutes incubation at 37° C., the assay mixture was filtered through a combination of GF-C and GF-B filters, pre-soaked in 0.05% Brij, using a Brandel cell harvester, and were washed three times using ice cold Tris-HCl buffer (50 mM). Radioactivity retained on the filters was determined by liquid scintillation counting. For each test compound, the concentration that inhibited binding of [³H]ketanserin by 50% was determined using curve fitting software (Prism). Kd values (concentration of ketanserin required to occupy 50% of the receptor binding sites at equlibrium)determined from saturation binding studies were then used to calculate inhibition dissociation constants (Ki) using the following equation:

$$Ki = \frac{{IC}_{50}}{1 + ( \frac{Radioligand concentration}{Radioligand Kd} )}$$

Membrane preparations from CHO-K1 cells expressing the cloned human 5-HT_{2C} receptor were obtained (Euroscreen). The membranes were thawed rapidly and diluted in assay buffer containing Tris-HCl (50 mM, pH 7.7), ascorbic acid (0.1%) and pargyline (10 μM). The membranes were resuspended by homogenisation, prior to adding 6 μg of membranes to assay wells containing [³H] mesulergine (1 nM), assay buffer (50 mM Tris at pH 7.7 and 0.1% ascorbic acid) containing pargyline (10 μM), and test compounds (1×10⁻¹⁰ to 1×10⁻⁴M). Non specific binding was determined in the presence of 100 μM mianserin. After 30 minutes incubation at 37° C., the assay mixture was filtered through a combination of GF-C and GF-B filters, pre-soaked in 1% bovine serum albumin, using a Brandel cell harvester, and were washed three times using ice cold Tris-HCl buffer (50 mM). Radioactivity retained on the filters was determined by liquid scintillation counting. For each test compound, the concentration that inhibited binding of [³H]mesulergine by 50% was determined using curve fitting software (Prism). Kd values (concentration of mesulergine required to occupy 50% of the receptor binding sites at equlibrium) determined from saturation binding studies were then used to calculate inhibition dissociation constants (Ki) using the following equation:

$$Ki = \frac{{IC}_{50}}{1 + ( \frac{Radioligand concentration}{Radioligand Kd} )}$$

The results are shown in table 1 below as pKi values.

TABLE 1

| Compound | 5-HT_{2B} | 5-HT_{2A} | 5-HT_{2C} |
|---|---|---|---|
| 1 | >6 | <5 | <6 |
| 2 | >7 | <6 | <6 |
| 3 | >6 | <6 | <6 |
| 4 | >5 | <5 | <5 |

Human Cloned 5-HT₂E Cell-Based Functional Assay

The following describes an in vitro functional assay using human cloned 5-HT_{2B} receptors to determine the ability of compounds to block the receptor.

CHO.K1 cells expressing cloned 5-HT_{2B} receptor were maintained In Ultra-CHO medium containing 400 μg/ml of G418, 100 U/ml penicillin, 100 μg/ml streptomycin, 2.5 μg/ml fungizone, in 95/5% O₂/CO₂ at 37° C. Ultra-CHO medium additionally supplemented with 1% foetal bovine serum was used when seeding the cells and removed after 5 hours. Cells were plated in Costar 96 well white, clear-bottomed plate at a density of 50,000 cells per well and incubated for at least 24 hours in 95/5% O₂/CO₂ at 37° C. before running the assay.

Media was removed from the wells and 200 μl of 4 μM Fluo-4 AM added, this was incubated in a Wallace Victor 2V workstation at 37° C. for 30 minutes. The Fluo-4 AM was then removed from the wells, which were then washed twice with 200 μl of buffer (HBSS without calcium/magnesium/phenol red, 20 mM HEPES, 1 mM Ca²⁺, 1 mM Mg²⁺, 2.5 mM probenecid, pH to 7.4), 180 μl of buffer or test compound was added to the well and incubated for 30 minutes. The Victor 2V injectors were used to inject 20 μl of 5-HT after obtaining 10 0.1-second baseline readings at 535 nm, followed by 150 readings.

All test compounds were aliquoted in 100% DMSO at 10 mM and diluted to 1 mM in 50% DMSO, subsequent dilutions were made using buffer. Buffer was also used to dilute the 5-HT. Data were analysed using Microsoft Excel and GraphPad Prism, with the latter used to produce sigmoidal dose-response curves for each compound. The compound concentration that inhibited the 5-HT response by 50% was taken (IC₅₀−M), and the results are shown in Table 2, as pIC₅₀, being the negative log (to the base 10) of the measured IC₅₀ values.

TABLE 2

| Compound | pIC₅₀ |
|---|---|
| 1 | >6 |
| 2 | >7 |
| 3 | >6 |
| 4 | >5 |

1. The use of a compound of formula I: 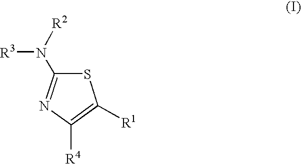
or a pharmaceutically acceptable salt thereof in the preparation of a medicament for the treatment of a condition alleviated by antagonism of a 5-HT_{2B} receptor, wherein
R¹ is selected from the group consisting of H, and optionally substituted C₁₋₆ alkyl, C₃₋₇ cycloalkyl, C₃₋₇ cycloalkyl-C₁₋₄ alkyl, and phenyl-C₁₋₄ alkyl;
R² and R³ are either:
(i) independently selected from H, R, R′, SO₂R, C(═O)R, (CH₂)ₙNR⁵R⁶, where n is from 1 to 4 and R⁵ and R⁶ are independently selected from H and R, where R is optionally substituted C₁₋₄ alkyl group, and R′ is an optionally substituted phenyl-C₁₋₄ alkyl group, or
(ii) together with the nitrogen atom to which they are attached, form an optionally substituted C₅₋₇ heterocyclic group;
R⁴ is an optionally substituted C₉₋₁₄ aryl group;
provided that when R¹ is H, at least two of the fused rings in R⁴ are aromatic.

2. The use according to claim 1, wherein R¹ is selected from H and optionally substituted C₁₋₆ alkyl and C₃₋₇ cycloalkyl

3. The use according to claim 1, wherein R² and R³ are independently selected from H, R and R′.

4. The use according to claim 1, wherein all of the fused rings in R⁴ are aromatic.

5. The use according to claim 1, wherein R⁴ is an optionally substituted C₉₋₁₄ carboaryl group.

6. The use according to claim 1, wherein R⁴ is a naphthyl group.

7. The use according to claim 1, wherein the conditions alleviated by antagonism of a 5-HT_{2B} receptor is a disorder of the GI tract.

8. A compound of formula I: 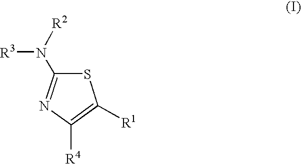
or a pharmaceutically acceptable salt thereof, for use in a method of therapy, wherein
R¹ is selected from the group consisting of H, C₁₋₆ alkyl optionally substituted by halo, hydroxy and amino, optionally substituted C₃₋₇ cycloalkyl, C₃₋₇ cycloalkyl-C₁₋₄ alkyl, and phenyl-C-₁₋₄ alkyl;
R² and R³ are either:
(i) independently selected from H, R, R′, SO₂R, C(═O)R, (CH₂)ₙNR⁵R⁶, where n is from 1 to 4 and R⁵ and R⁶ are independently selected from H and R, where R is a C₁₋₄ alkyl group optionally substituted by hydroxy, alkoxy and amido, and R′ is an optionally substituted phenyl-C₁₋₄alkyl group, or
(ii) together with the nitrogen atom to which they are attached, form an optionally substituted C₅₋₇ heterocyclic group;
R⁴ is an optionally substituted C₉₋₁₄ carboaryl group;
provided that when R¹ is H, R² and R³ are independently selected from H and R, and R⁴ is optionally substituted napth-1-yl.

9. The use according to claim 9, wherein R¹ is selected from H and optionally substituted C₁₋₆ alkyl and C₃₋₇ cycloalkyl

10. The use according to claim 8, wherein in R² and R³, R is an optionally substituted C₁₋₄ alkyl group.

11. The use according to claim 8, wherein R¹ is not H.

12. The use according to claim 11, wherein R² and R³ are independently selected from H, R and R′.

13. The use according to claim 11, wherein R⁴ is a napthy-1-yl group.

14. A pharmaceutical composition comprising a compound described in claim 8 or a pharmaceutically acceptable salt thereof together with a pharmaceutically acceptable carrier or diluent.

15. A compound of formula I: 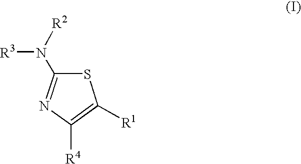
or a salt, solvate or chemically protected form thereof,
wherein
R¹ is CH(CH₃)₂;
R² and R³ are either:
(i) independently selected from H, R, R′, SO₂R, C(═O)R, (CH₂)ₙNR⁵R⁶ where n is from 1 to 4 and R⁵ and R⁶ are independently selected from H and R, where R is a C₁₋₄ alkyl group optionally substituted by hydroxy, alkoxy and amido, and R′ is an optionally substituted phenyl-C₁₋₄ alkyl group, or
(ii) together with the nitrogen atom to which they are attached, form an optionally substituted C₅₋₇ heterocyclic group;
R⁴ is an optionally substituted C₉₋₁₄ carboaryl group

16. A compound according to claim 15, wherein R² and R³ are independently selected from H, R and R′.

17. A compound according to claim 15, wherein R⁴ is a naphthyl group.

18. A method of treating a condition which can be alleviated by antagonism of a 5-HT_{2B} receptor, which method comprises administering to a patient in need of treatment an effective amount of a compound of formula I according to claim 1, or a pharmaceutically acceptable salt thereof.
